(19) United States
(12) Patent Application Publication
(10) Pub. No.: US 2006/0093593 A1
(43) Pub. Date: May 4, 2006

(54) Pro-biotic broad-spectrum nourishment preparation in powder, liquid, paste, or gas form

(76) Inventors: Jan Andrejkovic, Bohdanovce (SK); Marta Andrejkovicova, Bohdanovce (SK); Dominik Mojzis, Kosice (SK); Vladimir Snir, Kosice (SK)

Correspondence Address: DONN K. HARMS;PATENT & TRADEMARK LAW CENTER, SUITE 100, 12702 VIA CORTINA, DEL MAR, CA, 92014, US

(21) Appl. No.: 11/267,227

(22) Filed: Nov. 3, 2005

(30) Foreign Application Priority Data
Nov. 4, 2004 (SK) PUV 289-2004

(51) Int. Cl. A61K 35/74

(52) U.S. Cl. 424/93.45

(57) ABSTRACT

A pro-biotic broad-spectrum nourishment preparation for employment as a food additive. The preparation provides an improvement of extraction in the nourishment of animals and the increase of immunity against diseases when consumed by animals in their food. Favored formulas employ a number of food additives including one or a combination of skimmed dried milk, dried whey, maltodextrine, fructooligasacharide, supernatant, Lactobacillus plantarum micro-organisms, and vitamins.

### FIELD OF TECHNOLOGY

The technical solution relates to a pro-biotic broad-spectrum nourishment preparation in powder, liquid, paste, or gas form, designed as a food additive for the improvement of extraction in the nourishment of animals and the increase of immunity against diseases.

### RECENT STATE OF TECHNOLOGY

Up to now pro-biotic preparations are employed on the basis of several stems of lactobaciles as part of milk food mixture.

A well-known pro-biotic preparation is Lactifern premix L-5 designed for breeding youngsters and nourishing farm animals, of which active elements is stabilized culture of living bacteria of milk fermentation of streptococcus faecium M 74 germs, that is internationally registered in the Czech Collection of Micro-organisms MU in Brno (CCM 6266), that decreases the level of endogenous bacterial stresses and stimulates natural immune and metabolic functions of the animals' organism. The disadvantage of this solution is the minimal improvement of production indicators, as are weight growth, little stimulation of growth, conversion of the feed or improvement of extraction in the nourishment of animals.

Another known pro-biotic preparation is Lactifern super-concentrate L-400, that is designed for spray application for cold granulated feed, of which active element is stabilized living culture of lactacidogenic bacteria of enterococcus faeciu M 74 germ, that is internationally registered in the Czech Collection of Micro-organisms MU in Brno (CCM 6226), that decreases the endogenous mocro-bial load and empowers intestinal protection bacteria and harmonizes metabolic functions. The disadvantage of this solution is the minimal improvement of production indicators of breeding, as are weight growth, little growth stimulation, conversion of the feed or improvement of extraction in the nourishment of animals.

Another known pro-biotic preparation is Lactiferm paste FE a. u. v., that is as a paste enriched with Fe ions for peroral treatment of youngsters, of which active element is stabilized living culture of lactacidogenic bacteria of enterococcus faeciu M 74 germs, that is internationally registered in the National Collection of Industrial Bacterial under registration No. NCIB 11 181, that effectively effects intestinal micro-flora of youngsters in the early breeding period and decreases the symptoms of anemia. The disadvantage of this solution is the minimal improvement of production indicators of breeding, as are weight growth, little growth stimulation, conversion of the feed or improvement of extraction in the nourishment of animals.

Another known solution is described in the published Slovak application of the patent PP No. 1 827-2002 titled “Application of Lactobacillus casei in immuno-stimulating peptides”, that is related to the usage of bacterial stem of Lactobacillus casei type for the preparation of mixture that is applied per os for the reinforcement of specific system immune response against pathogenous micro-organism. The mentioned mixture can be mainly a food-stuff or a food additive. The disadvantage of this invention is only the unilateral application for increasing immunity, without remarkable improvement of production breeding indicators, as are weight growth, little growth stimulation, conversion of the feed or improvement of extraction in the nourishment of animals.

Another known solution is described in the published Slovak application of the patent PP No. 738-2002 titled “Vaccine for the treatment of prostate inflammation and benign hyperplasia of prostate including stems of Lactobacillus genus”, where the described vaccine for the treatment of prostate inflammation and benign hyperplasia of prostate (1st and 2nd stage) including stems of Lactobacillus genus, that have protecting effects against Lactobacillus genus resulting necrosis of cells, in inactivated form and carrying materials and/or protecting materials normally used in vaccine preparations. Later the usage of stems of Lactobacillus genus for the production of vaccines able to treat prostate inflammation and benign hyperplasia of prostate (1st and 2nd stage) is described. The disadvantage of this invention is only the unilateral application for treatment of prostrate inflammation without remarkable improvement of production breeding indicators, as are weight growth, little growth stimulation, conversion of the feed or improvement of extraction in the nourishment of animals.

Another known solution is described in the published Slovak application of the patent PP No. 163-2002 titled “Tablet Including at least one Genus of Pro-biotic Micro-organism and the Method of its Production”, where the tablet that includes at least one genus of pro-biotic micro-organism is described, while it itself comprises pro-biotic micro-organism, has at least one enteric coating, has increased active pro-biotic micro-organisms in human and/or animal digestion system and consequently increased effect on health. The disadvantage of this invention is only the unilateral application of the tablet, for the treatment of prostate without remarkable improvement of production breeding indicators, as are weight growth, little growth stimulation, conversion of the feed or improvement of extraction in the nourishment of animals.

Another known solution is described in the published Slovak application of the patent PP No. 435-2002 titled “Lactobacillus helveticus Bacteria Producing Lactacid with High Production of Tripeptide, Fermented Milk Product and Method of its Production”, where the fermented milk product including bacteria producing lactacid able to produce a large number of lactoripeptide and a large number of active substance with hypotensive activity and anti-stress effect, and that can be used in form of foodstuff or drinks. Lactobacillus helveticus bacteria producing lactacid has specific bacteriological features, while these bacteria, if cultivated in animal milk media including 9% weight of dry matter of nonfat milk, produce tripeptide Val-Pro-Pro and Ile-Pro-Pro in amount 60 mg. According to the content of Val-Pro-Pro on ml of media and these bacteria react with extra-cellular protease activity not lower than 400 U/OD590. The described fermented milk product is gained from the fermented animal milk from these bacteria producing lactacid. The disadvantage of this invention is only the unilateral application with anti-stress effect without remarkable improvement of production breeding indicators, as are weight growth, little growth stimulation, conversion of the feed or improvement of extraction in the nourishment of animals

Another known solution is described in the published Slovak application of the patent PP No. 644-2002 titled “Potented Pro-biotic Food Additive for Suckers and Weanlings”, where a combined pro-biotic preparation with potented pro-biotic effect applied in the entire digestive system of animals comprising of Lactobacillus casei pro-biotic culture and potented components of oligosacharides FOS and maltodextrines KMS X-70 is described. The disadvantage of this invention is the minimal improvement of production breeding indicators, as are weight growth, little growth stimulation, conversion of the feed or improvement of extraction in the nourishment of animals

Another known solution is described in the published Slovak application of the patent PP No. 645-2002 titled “Potented Pro-biotic Food Additive for Calves”, where a combined pro-biotic preparation with potented pro-biotic effect applied in the entire digestive system of calves comprising of Lactobacillus casei pro-biotic culture and potented component maltodextrines KMS X-70 is described. The disadvantage of this invention is the minimal improvement of production breeding indicators, as are weight growth, little growth stimulation, conversion of the feed or improvement of extraction in the nourishment of animals.

Another known technical solution is described in the registered Slovak Utility Model UVz No. 3 505 titled “Potented Pro-biotic Food Additive for Suckers and Weanlings”, where it is stated that it consists of 32 weight parts of pro-biotic stem of Lactobacillus casei micro-organism with 10⁸ germ density, 18 weight parts of fructoligosacharides, and 50 weight parts of maltodextrine. The disadvantage of both pro-biotic food additives is their limited application for the prevention of diarrheal disease. Another disadvantage is the minimal improvement of production breeding indicators, as are weight growth, little growth stimulation, conversion of the feed or improvement of extraction in the nourishment of animals.

Another known technical solution is described in the registered Slovak Utility Model UVz No. 3 506 titled “Potented Pro-biotic Food Additive for Calves”, where the technical solution is introduced, which essence is that it is comprised of 40 weight parts of pro-biotic stem of lactobacillus casei micro-organism with 1,10⁸ germ density, 60 weight parts of maltodextrine. The disadvantage of both pro-biotic food additives is their limited application for the prevention of diarrheal disease. Another disadvantage is the minimal improvement of production breeding indicators, as are weight growth, little growth stimulation, conversion of the feed or improvement of extraction in the nourishment of animals.

Another known technical solution is described in the registered Slovak Utility Model UVz No. 3 509 titled “Potented Pro-biotics”, where the technical solution is introduced, which essence is that potented pro-biotics are comprised of 26 weight parts of pro-biotic stem of lactobacillus casei micro-organism with 1,10⁸ germ density, 12 weight parts of fructooligosacharides, 38 weight parts of maltodextrine, 14 weight parts of fish oil, and 10 weight parts of aloe gel. The disadvantage of both technical solutions is their limited application for the prevention diarrheal disease. Another disadvantage is the minimal improvement of production breeding indicators, as are weight growth, little growth stimulation, conversion of the feed or improvement of extraction in the nourishment of animals.

Another known technical solution is described in the registered Slovak Utility Model UVz No. 3 744 titled “Pro-biotic Food Additive for Poultry and Rabbits” which technical solution comprises 40 weight parts of Enterococcus faecium EK 13 stem with 1.0×10⁹ germ density (KTJ/ml), with production of stable anti-microbe substance—enterocine A (pepid of molecular weight 4 829 Da) with inhibition effect on unwanted bacteria including Escherichia coli and some of other ones of Salmonella and Listeria genuses and 60 weight parts of dried skimmed milk. The disadvantage of this technical solution is its unilateral application on the prevention of diseases and support of the growth of the poultry. Another disadvantage is the minimal improvement of production breeding indicators, as are weight growth, little growth stimulation, conversion of the feed or improvement of extraction in the nourishment of animals In the Slovak Patent File P No. 283 008 titled “Biologically clean Culture Lactobacillus Rhamnosus, Composition with its Content and its Application”, is described the biologically clean culture L. rhamnosus HN 001 AGAL, depository No. NM97/09514 or L. rhamnosus HN067 AGAL depository No. NM97/01925 having the ability to increase immunity of the sucker, or its mutants and variants that retain the mentioned ability. The composition of this biologically clean culture consists of one or more cultures selected from L. rhamnosus HN001 or L. rhamnosus HN067, B. lactis HN019 AGAAL with depository No. NM97/09515 in immuno-stimulating concentration with physiologically acceptable excipient or solvent. This biologically clean culture or composition with its content is used for the preparation of medicaments for increasing natural and gained immunity. The disadvantage of this invention protected by patent is its unilateral application for increasing immunity. Another disadvantage is the minimal improvement of production breeding indicators, as are weight growth, little growth stimulation, conversion of the feed or improvement of extraction in the nourishment of animals.

In the Slovak Patent File P No. 280 832 titled “Instrument on the Basis of Pro-biotics, Method of Preparation of Pro-biotic Bacteria present in the Instrument and Application of this Instrument, Instrument on the basis of Pro-biotics”, of which essence lies in the fact that it is created by lyofilizated pro-biotic bacteria of milk ferment of Enterococus or Bifidobacterium genuses with organically bound selenium in amount 410-420 ug Se/lg of lyofilizated bacteria is described, while the content of living germs in 1 g of lyofilizated bacteria is 2.3-9.0. 10 exp(11). Pro-biotical bacteria included in the instrument are prepared in such a way that bacteria of milk fermentation of Entercococcus and Bifidobacterium germs are cultivated in a cultivation media, adding anorganic salt of selenium in amount 0.00033-0.00046% at temperature 37-42° C. during 15-18 hours, the gained product is separated, is rinsed in sterile saline solution and finally it is lyofilizated. The disadvantage of this instrument is that it can be used only for the preparation of food-stuff products with pro-biotic effects. Another disadvantage is the minimal improvement of production breeding indicators, as are weight growth, little growth stimulation, conversion of the feed or improvement of extraction in the nourishment of animals.

In the Slovak Patent File P No. 281 476 titled “Pro-biotic Preparation for Youngsters Chewers” and in Utility Model UVz No. 1 060 titled “Pro-biotic Preparation for Youngsters Chewers” is described a preparation for the prevention of diarrheal diseases, that in lyofilizated form consists of 1.10 exp(8)-1.10 exp(9) of Lactobacillus casei sbp. pseudoplantarum germs in 1 g of dried skimmed milk. Their disadvantage is that they have effect not only on the development of digestive functions but also on the immune system. Another disadvantage is the minimal improvement of production breeding indicators, as are weight growth, little growth stimulation, conversion of the feed or improvement of extraction in the nourishment of animals.

The Essence of Technical Solution

The introduced disadvantages to a substantial rate removes pro-biotic broad-spectrum nourishment preparation in powder, liquid, paste, or gas form, assigned mainly as a food additive, for the improvement of extraction in the feeding of animals and increasing immunity against diseases, according to the technical solution, of which essence lies in the fact that it consists of up to 97.5% of binding media, 0.1-2% potented additional element, 0.001-0.5% of supporting element and bacterial stems with 10⁴-10¹² genuses in 1 gram of binding media.

It is suitable that the pro-biotic broad-spectrum nourishing preparation in powder, liquid, paste, or gas form consists of up to 30% addition element, that is supermatant.

It is suitable that the pro-biotic broad-spectrum nourishment preparation in powder, liquid, paste, or gas form consists of up to 3% addition element, that is compressed air.

It is suitable that the bacterial stems are created by 1-100% of lactobacillus plantarum microorganisms with depository No. CCM 7102, deposited in the International Depository—Czech Collection of Microorganisms MU in Brno, Czech Republic, and/or 1-100% of lactobacillus fermentum 2I₃ CCM 7158 microorganisms, deposited in the International Depository—Czech Collection of Microorganisms MU in Brno, Czech Republic and/or or 1-100% of lactobacillus casei sbp pseudoplantarum 249/89 CCM 7159 microorganismsm, deposited in the International Depository—Czech Collection of Microorganisms MU in Brno, Czech Republic and/or or 1-100% of lactobacillus acidophilus 3/99 microorganisms, deposited in the International Depository—Czech Collection of Microorganisms MU in Brno, Czech Republic and/or or 1-100% of lactobacillus casei microorganisms 20/97, deposited in the International Depository—Czech Collection of Microorganisms MU in Brno, Czech Republic and/or or 1-100% of lactobacillus plantarum 5/95 microorganisms, deposited in the International Depository—Czech Collection of Microorganisms MU in Brno, Czech Republic and/or or 1-100% of lactobacillus plantarum 18/96 microorganisms, deposited in the International Depository—Czech Collection of Microorganisms MU in Brno, Czech Republic and/or or 1-100% of lactobacillus plantarum 4/97 microorganisms, deposited in the International Depository—Czech Collection of Microorganisms MU in Brno, Czech Republic and/or or 1-100% of lactobacillus plantarum 15/97 microorganisms, deposited in the International Depository—Czech Collection of Microorganisms MU in Brno, Czech Republic and/or or 1-100% of lactobacillus plantarum 16/97 microorganisms, deposited in the International Depository—Czech Collection of Microorganisms MU in Brno, Czech Republic and/or or 1-100% of lactobacillus plantarum 13/98 microorganisms, deposited in the International Depository—Czech Collection of Microorganisms MU in Brno, Czech Republic and/or or 1-100% of lactobacillus plantarum 94/98 microorganisms, deposited in the International Depository—Czech Collection of Microorganisms MU in Brno, Czech Republic and/or or 1-100% of lactobacillus plantarum 2/99 microorganisms, It is suitable that the binding medium is created by 10-100% vehiculum and/or 10-100% of dried whey and/or 10-100% of skimmed dry milk.

It is suitable that the potenting additive element is created by 10-100% of maltodextrine and/or 10-100% of fructooligosacharide.

It is suitable that the supporting element is created by enzymes, vitamins, and micro-elements of Fe, Zn, Mg, Cu, Ca, P, K, Se, I, Na.

### EXAMPLES OF EXECUTION OF TECHNICAL SOLUTION

### Example No. 1

Pro-biotic broad-spectrum nourishment preparation in powder form, according to the technical solution, includes 32.5 g skimmed dried milk, 65 g dried whey, 1 g matlodextrine, 1 g fructooligosacharide, microorganisms Lactobacillus plantarum, with depository No. CCm 7102, granted by International Depository—Czech Collection of micro-organisms MU in Brno, Czech Republic, with density 10⁸ in 1 gram of skimmed dried milk and dried whey and 0.5 g enzymes, vitamins, Fe, Zn, Mg, Cu, Ca, P, K, Se, I, Na micro-elements.

### Example No. 2

Pro-biotic broad-spectrum nourishment preparation in dried form, according to the technical solution, includes 487.5 g of skimmed dried milk, 5 g of maltodextrine, 5 g of fructooligasacharide, micro-organisms Lactobacillus plantarum 5/95, deposited in the Czech Collection of micro-organisms with density 10⁸ in 1 gram of skimmed dried milk and pro-biotic stems of micro-organisms Lactobacillus acidophilus 3/99, deposited in the Czech Collection of Micro-organisms MU in Brno, Czech Republic with bacteria density 10¹² in 1 gram of skimmed dried milk and dried whey and 2.5 g of enzymes, Fe, Zn, Mg, Cu, Ca, P, K, Se, I, Na micro-elements.

### Example No. 3

Pro-biotic broad-spectrum nourishment preparation in paste form, according to the technical solution, includes 405 g of skimmed dried milk, 350 g of dried whey, 225 g of vehiculum, 7.5 g of maltodextrine, 7.5 g of fructooligasacharide, micro-organisms Lactobacillus plantarum 18/96, deposited in the Czech Collection of micro-organisms MU in Brno, Czech Republic, with density 10⁷ in 1 gram of skimmed dried milk and dried whey and vehiculume that creates a form that can be spread, micro-organisms Lactobacillus fermentum 2,3 with deposit No. CCM 7158, granted by the International Depository—Czech Collection of Micro-organisms MU in Brno, Czech Republic with bacteria density 10¹⁰ in 1 gram of skimmed dried milk and dried whey and vehiculume, micro-organisms Lactobacillus casei 20/97, deposited by the Czech Collection of micro-organisms MU in Brno, Czech Republic with bacteria density 10¹⁰ in 1 gram of skimmed dried milk and dried whey and vehiculume, and 5 gram enzyme, vitamins and Fe, Zn, Mg, Cu, Ca, P, K, Se, I, Na micro-elements.

### Example No. 4

Pro-biotic broad-spectrum nourishment preparation in paste form, according to the technical solution, includes 725 g of skimmed dried milk, 757 g of dried whey, 475 g of vehiculum, 15.5 g of maltodextrine, 20 g of fructooligasacharide, micro-organisms Lactobacillus casei 294/89, with deposit No CCM 7159, granted by the International Depository—Czech Collection of Micro-organisms MU in Brno, Czech Republic with density 10⁷ in 1 gram of skimmed dried milk and dried whey and vehiculume, micro-organisms Lactobacillus plantarum 4/97, deposited by the Czech Collection of micro-organisms MU in Brno, Czech Republic with bacteria density 10¹⁰ in 1 gram of skimmed dried milk and dried whey and vehiculume, micro-organisms Lactobacillus plantarum 15/97, deposited in the Czech Collection of micro-organisms MU in Brno, Czech Republic, with bacteria density 10¹⁰ in 1 gram of skimmed dried milk and dried whey and vehiculume, and 7.5 g of enzymes, vitamins, and Fe, Zn, Mg, Cu, Ca, P, K, Se, I, Na micro-elements

### Example No. 5

Pro-biotic broad-spectrum nourishment preparation in gas form, according to the technical solution, includes 78 g of skimmed dried milk, 110 g of dried whey, 2 g of maltodextrine, 2 g of fructooligasacharide, 50 g supernatant, 7 g compressed air, Lactobacillus plantarum micro-organisms 16/96, deposited in the Czech Collection of micro-organisms MU in Brno, Czech Republic, with density 10¹⁰ in 1 gram of skimmed dried milk and dried whey and Lactobacillus plantarum micro-organisms 13/98, deposited in the Czech Collection of micro-organisms MU in Brno, Czech Republic with bacteria density 10¹⁰ in 1 gram of skimmed dried milk and dried whey and 1 gram of enzyme, vitamins and Fe, Zn, Mg, Cu, Ca, P, K, Se, I, Na micro-elements.

### Example No. 6

Pro-biotic broad-spectrum nourishment preparation in liquid form, according to the technical solution, includes 134 g of skimmed dried milk, 325 g of dried whey, 6 g of maltodextrine, 1 g of fructooligasacharide, 32 g supernatant, that is a separated element of the biomass at production, micro-organisms Lactobacillus plantarum 94/98, deposited in the Czech Collection of micro-organisms MU in Brno, Czech Republic, with density 10¹⁰ in 1 gram of skimmed dried milk and dried whey and Lactobacillus plantarum micro-organisms 2/99, deposited in the Czech Collection of micro-organisms MU in Brno, Czech Republic with bacteria density 10¹⁰ in 1 gram of skimmed dried milk and dried whey and 2 gram of enzyme, vitamins and Fe, Zn, Mg, Cu, Ca, P, K, Se, I, Na micro-elements.

Industrial Efficiency

Technical solution can be used as pro-biotic veterinary preparation, in powder form after centrifugal and lyophilisation form, using vacuum and freeze cca −50° C.-−70°, in liquid form, when the thinner agent is water, in gas form, that is the combination of liquid form with using fuel gas or spray, in form of paste that can be spread in agriculture, breeding, veterinary medicine, food industry, when optimizing nourishment, for the improvement of production in animal nourishment, growth stimulation and improvement of food utilizing, for the prevention of diarrheal diseases of bacterial reason, for fast inoculation of digestion apparatus of young animals for the prevention of infectious diseases of animals, for re-colonization of intestinal micro-flora after antibiotics and chemotherapeutic treatment, for the improvement and support of immune reactions of animals. The technical solution can also be used in humans.

What is claimed is:

1. Pro-biotic broad-spectrum nourishment preparation in powder, liquid, paste or gas form, assigned mainly as a food additive for improving production in animal nourishment and increasing immunity against diseases, that is characterized by the fact that it comprises 97.5% wt. binding material, 0.1-0.2% wt. of potentiating additional element, 0.001-0.5% wt. of supporting element and bacterial stems with germ density 10⁴-10¹² in 1 gram of binding material.

2. Pro-biotic broad-spectrum nourishment preparation in powder, liquid, paste, or gas form, according to requirement 1 characterised by the fact that it comprises up to 30% wt. of additive element that is supernatant.

3. Pro-biotic broad-spectrum nourishment preparation in powder, liquid, paste, or gas form, according to requirement 1 and 2 characterized by the fact that it comprises up to 3% wt. of additive elements, that is compressed air.

4. Pro-biotic broad-spectrum nourishment preparation in powder, liquid, paste, or gas form, according to requirement 1 and 3, characterised by the fact that the bacterial stems are created by 1-100% wt. of microorganisms lactobacillus plantarum with depository No. CCM 7102 and/or 1-100% wt. of micro-organisms lactobacillus fermentum 2I3 with depository No. CCM 7158 and/or 1-100% wt. of microorganismsm lactobacillus casei sbp. pseudoplantarum 294/89 with depository No. CCM 7159 and/or 1-100% wt. of microorganismsm lactobacillus casei sbp. pseudoplantarum 294/89 with depository No. CCM 7159, and/or 1-100% wt. of lactobacillus acidophilus micro-organisms 3/99, and/or 1-100% wt. of lactobacillus casei micro-organisms 20/97, and/or 1-100% wt. of lactobacillus plantarum micro-organisms 5/95, and/or 1-100% wt. of lactobacillus plantarum micro-organisms 18/96, and/or 1-100% wt. of lactobacillus plantarum micro-organisms 4/97, and/or 1-100% wt. of lactobacillus plantarum micro-organisms 15/97, and/or 1-100% wt. of lactobacillus plantarum micro-organisms 16/97, and/or 1-100% wt. of lactobacillus plantarum micro-organisms 13/98, and/or 1-100% wt. of lactobacillus plantarum micro-organisms 94/98, and/or 1-100% wt. of lactobacillus plantarum micro-organisms 2/99.

5. Pro-biotic broad-spectrum nourishment preparation in powder, liquid, paste, or gas form, according to requirements 1-4, characterised by the fact that the binding medium is from 10-100% wt. of vehiculum and/or 1-100% wt. of dried whey and/or 1-100% wt. of skimmed dried milk.

6. Pro-biotic broad-spectrum nourishment preparation in powder, liquid, paste, or gas form, according to requirements 1-5, characterized by the fact that the patenting element is from 10-100% wt. of maltodextrine and/or 1-100% wt. of fructooligosacharide.

7. Pro-biotic broad-spectrum nourishment preparation in powder, liquid, paste, or gas form, according to requirements 1-6, characterized by the fact that the binding medium is from enzymes, vitamins, and micro-elements of Fe, Zn, Mg, Cu, Ca, Pa, K, Se, I, Na.
